(19) United States
(12) Patent Application Publication
(10) Pub. No.: US 2018/0071076 A1
(43) Pub. Date: Mar. 15, 2018

(54) Aneurysm Treatment Method

(71) Applicant: Lifetech Scientific (Shenzhen) Co., Ltd., Shenzhen (CN)

(72) Inventors: Wei Guo, Shenzhen (CN); Deyuan Zhang, Shenzhen (CN); Benhao Xiao, Shenzhen (CN); Wuyang Li, Shenzhen (CN); Caiping Liu, Shenzhen (CN)

(73) Assignee: Lifetech Scientific (Shenzhen) Co., Ltd., Shenzhen (CN)

(21) Appl. No.: 15/241,349

(22) Filed: Sep. 13, 2016

(51) Int. Cl. A61F 2/07; A61F 2/954

(52) U.S. Cl. CPC A61F 2/07; A61F 2002/067; A61F 2/954

(57) ABSTRACT

A method for treating an aneurysm with at least one branch blood vessel branched from a diseased aorta comprises implanting an aortic stent graft and an auxiliary stent into a diseased blood vessel to isolate the aneurysm, wherein the auxiliary stent comprises a bifurcated stent graft, one end of the aortic stent graft is attached to the wall of a healthy blood vessel upstream of the aneurysm, and the auxiliary stent comprises a body portion and a branch portion communicated with the body portion, one end of the body portion is communicated with a healthy blood vessel downstream of the aneurysm, the other end of the body portion is communicated with the aortic stent graft, and the branch portion is suspended within the diseased blood vessel. A straight stent graft is advanced towards the branch portion from the location downstream of the bifurcated stent graft and deployed to communicate the branch portion and the branch blood vessel.

### TECHNICAL FIELD

The present invention relates to a vascular interventional treatment technique, and more particularly, to a method for treating an aneurysm using stent grafts.

### BACKGROUND ART

After an artery blood vessel is degenerated and/or subjected to trauma, a blood flow may cause the diseased blood vessel to expand abnormally, thereby forming an aneurysm. Both the bending and compression performances of the diseased blood vessel at the aneurysm are inferior to those of a healthy blood vessel. Under the impact of the blood flow, the diseased blood vessel is prone to be broken, which leads to massive internal hemorrhage before long, thereby endangering the life of the patient.

For the treatment of an aneurysm, a stent graft is usually implanted into the diseased blood vessel to isolate the aneurysm, so that the blood flows through the tube cavity of the stent graft to prevent the aneurysm from being broken due to impact. However, if a branch blood vessel for blood supply to visceral organs branches out at the aneurysm, the stent graft will also isolate the branch blood vessel, so that the blood supply to the corresponding visceral organs is cut off. For example, if the stent graft cuts off the blood supply to the renal artery or inferior mesenteric artery, intestinal necrosis, liver and kidney function failure or other complications may be caused. Therefore, at the same time the aneurysm is treated, there is a need for reconstruction of the blood supply of any branch blood vessel that branches from the aneurysm, so as to prevent the blood flow of the branch blood vessel from being cut off.

### SUMMARY OF THE INVENTION

The present invention provides a method for treating an aneurysm in a diseased aorta, which has at least one branch blood vessel branched from the diseased aorta blood vessel. If the aneurysm involves the downstream branch blood vessel, the treatment method comprises successively implanting an aortic stent graft and an auxiliary stent into the diseased blood vessel, to isolate the aneurysm and also to establish blood supply for branch blood vessels in the diseased blood vessel using the branch portion of the auxiliary stent under the combined action of the two stents. The auxiliary stent may be a bifurcated stent graft including a body portion and a branch portion communicated with the body portion.

Before the bifurcated stent graft is implanted, the aortic stent graft should be implanted into the diseased blood vessel from a location upstream of the aneurysm in the first place. The aortic stent graft may be an abdominal aortic stent graft, or a thoracic aortic stent graft, or an aortic arch stent graft. After implantation, one end of the aortic stent graft is communicated with a healthy blood vessel upstream of the aneurysm, and the aortic stent graft has an open end suspended within the diseased blood vessel.

The implantation of the bifurcated stent graft may be carried out according to the following steps: introducing a first guide wire into the diseased blood vessel and the aortic stent graft successively from the location downstream of the aneurysm, advancing a first catheter delivery system provided with the bifurcated stent graft over the first guide wire in the aorta from the location downstream of the aneurysm toward the location upstream of the aneurysm, and releasing the bifurcated stent graft such that the branch portion is deployed within the diseased blood vessel.

At this moment, the first guide wire will pass through the body portion of the bifurcated stent graft, one end of the body portion of the bifurcated stent graft is attached to the wall of a healthy blood vessel downstream of the aneurysm, the other end of the body portion is communicated with the aortic stent graft to isolate the aneurysm, meanwhile, the branch portion is suspended within the diseased blood vessel, with its suspended end pointing to the location upstream of the aneurysm, and is isolated from the branch blood vessel, to facilitate the subsequent establishment of blood supply between the branch portion and the branch blood vessel.

Then, a straight stent graft can be advanced towards the branch portion from a location downstream of the bifurcated stent graft. The straight stent graft is deployed to communicate the branch portion and the branch blood vessel, thereby establishing blood supply between the aorta and the branch blood vessel.

The body portion of the bifurcated stent graft may be directly muff-coupled with the aortic stent graft to achieve the isolation of the aneurysm. If the body portion of the bifurcated stent graft is too short to be directly connected with the suspended open end of the aortic stent graft, the auxiliary stent can be the combination of a bifurcated stent graft and a cuff stent, that is, the cuff stent is muff-coupled between the body portion of the bifurcated stent graft and the aortic stent graft, so that the aortic stent graft and the bifurcated stent graft are communicated by the cuff stent, thus achieving the isolation of the aneurysm.

When there is a need for introducing a cuff stent graft to communicate the aortic stent graft, the treatment method comprises advancing at least one cuff straight stent graft along the body portion from the location downstream of the bifurcated stent graft and deploying it, so that the two ends of the cuff stent graft are muff-coupled with the open end of the aortic stent graft and the open end of the body portion, respectively.

Before advancing the straight stent graft, the first guide wire can be pulled out from the aortic stent graft, such that the first guide wire enters the branch blood vessel from the branch portion; then, a second catheter delivery system provided with the straight stent graft is advanced over the first guide wire in the aorta from the location downstream of the aneurysm toward the location upstream of the aneurysm. That is, the same guide wire can be used for establishing advance paths for the bifurcated stent graft and the straight stent graft.

Optionally, before advancing the straight stent graft, the first guide wire can be removed from the human body and a second guide wire is introduced into the aorta from the location downstream of the aneurysm, such that the second guide wire enters the branch blood vessel through the branch portion; then, a second catheter delivery system provided with the straight stent graft is advanced over the second guide wire in the aorta from the location downstream of the aneurysm toward the location upstream of the aneurysm.

When more than two branch blood vessels are branched from the diseased aorta, a bifurcated stent graft having more than two branch portions can be applied, and then more than two straight stent grafts are implanted according to the aforementioned method such that each straight stent graft is communicated with one branch blood vessel and one branch portion, respectively.

Optionally, when more than two branch blood vessels are branched from the diseased aorta, more than two bifurcated stent grafts and more than two straight stent grafts can be applied, and each bifurcated stent graft has only one branch portion. These straight stent grafts are implanted such that each bifurcated stent graft is communicated with one straight stent graft and the body portions of the plurality of bifurcated stent grafts are mutually connected in series and directly communicated with the aortic stent graft.

If the aneurysm does not involve the downstream branch blood vessel, the treatment method comprises implanting an aortic stent graft into a diseased blood vessel to isolate the aneurysm, two ends of which are respectively attached to the wall of a healthy blood vessel upstream and downstream of the aneurysm, a branch portion of the aortic stent graft is suspended within the diseased blood vessel. A straight stent graft is then advanced towards the branch portion from the location downstream of the bifurcated stent graft, and deployed to communicate the branch portion and the branch blood vessel.

Alternatively, after the aneurysm is isolated with the aortic stent graft, the method comprises advancing a bifurcated stent graft towards the branch portion from the location downstream of the aortic stent graft, deploying the bifurcated stent graft, and then advancing and deploying the straight stent graft to communicate the branch portion and the branch blood vessel.

If more than two branch blood vessels are branched from the diseased aorta, and the aortic stent graft comprises more than two branch portions, then the treatment method comprises implanting more than two straight stent grafts, with each straight stent graft communicated with one branch blood vessel and one branch portion, respectively.

Alternatively, the aortic stent graft comprises a branch portion, the bifurcated stent graft comprises more than two branch portions, and the method comprises implanting more than two straight stent graft, each of which is respectively communicated with a branch blood vessel and one of the branch portions of the bifurcated stent graft.

### BRIEF DESCRIPTION OF THE DRAWINGS

The present invention will be further illustrated hereinafter with reference to the accompanying drawings and embodiments, in which the drawings are as follows:

FIG. 1 is a schematic view of an aortic stent graft provided by an embodiment of the present invention:

FIG. 2 is a schematic view of a bifurcated stent graft provided by an embodiment of the present invention:

FIG. 3 is a schematic view of a straight stent graft provided by an embodiment of the present invention:

FIG. 4 is a schematic view of an aortic stent graft deployed within the aorta in an embodiment of the invention:

FIG. 5 is a schematic view showing the introduction a first guide wire after the aortic stent graft of FIG. 4 is deployed:

FIG. 6 is a schematic view of a bifurcated stent graft deployed within the aorta of the invention:

FIG. 7 is a schematic view showing the introduction of a second guide wire after the bifurcated stent graft is deployed as shown in FIG. 6:

FIG. 8 is a schematic view of a straight stent graft deployed within the aorta of the invention:

FIG. 9 is a schematic view of a reconstructed blood supply:

FIG. 10 is a schematic view of an aortic stent graft and a bifurcated stent graft deployed within the aorta in another embodiment of the invention:

FIG. 11 is a schematic view of a cuff stent graft deployed within the aorta:

FIG. 12 is a schematic view of a straight stent graft deployed within the aorta; and

FIG. 13 is a schematic view of reconstruction of the blood supply for multiple branch blood vessels in still another embodiment of the invention.

FIG. 14 is a schematic view of a reconstructed blood supply for multiple branch blood vessels in yet another embodiment of the invention.

FIG. 15 is a schematic view of a bifurcated stent graft provided by an embodiment of the present invention.

FIG. 16 is a schematic view showing the reconstruction of blood supply for three branch blood vessels.

### DETAILED DESCRIPTION OF THE INVENTION

in order to more clearly understand the technical features, purposes and effects of the present invention, the embodiments of the invention will now be described in detail with reference to the accompanying drawings. The following embodiments are described by taking the abdominal aorta for instance, which are illustrative of the invention only and should not to be construed as limiting the invention. In each drawing, the blood flow direction is indicated by an arrow, and the arrow points from the upstream to the downstream of the aorta. Each drawing is merely illustrative of the distribution of blood vessels, and cannot accurately represent the actual locations of each blood vessel due to individual differences.

In condition of the aneurysm involving downstream branch blood vessel, the method for treating an aneurysm provided by the present invention comprises: first implanting an aortic stent graft into the diseased blood vessel from a location upstream of the aneurysm, and deploying the stent graft such that one end of it adheres to the wall of a healthy blood vessel upstream of the aneurysm and the other end is suspended within the diseased blood vessel; then, implanting a bifurcated stent graft into the diseased blood vessel from a location downstream of the aneurysm, wherein the bifurcated stent graft comprises a body portion and a branch portion, and after deploying the bifurcated stent graft, one end of the body portion is communicated with a healthy blood vessel downstream of the aneurysm and the other end is connected with the suspended end of the aortic stent graft to isolate the aneurysm, and the branch portion is suspended within the diseased blood vessel; then, a straight stent graft (i.e. a stent graft comprising no branch) is advanced within the body portion from a location downstream of the bifurcated stent graft towards the branch portion to connect the branch portion and the branch blood vessel.

The aortic stent graft may be a straight tube-shaped thoracic aortic stent graft, or an abdominal aortic stent graft having a long branch and a short branch, or an aortic arch stent graft having a plurality of branches. If the bifurcated stent graft is short, after implantation, the end of the body portion located within the diseased blood vessel may not be directly connected with the suspended end of the aortic stent graft; for example, the thoracic aortic stent graft. At this moment, a straight tube-shaped cuff stent graft is advanced, from a location downstream of the aneurysm, towards the suspended end along the body portion, and the cuff stent graft is communicated with the body portion and the suspended end to insulate the aneurysm.

Alternatively, as an alternative of the aforementioned method, prior to the implantation of the bifurcated stent graft, the straight tube-shaped cuff stent graft can be implanted such that one end of the cuff stent graft is attached to the inner wall of a healthy blood vessel downstream of the aneurysm and the other end is suspended within the diseased blood vessel, then the bifurcated stent graft is implanted such that one end of the body portion of the bifurcated stent graft is attached to the inner wall of the suspended end of the cuff stent graft and the other end adheres to the inner wall of the aortic stent graft, so as to insulate the aneurysm.

Similarly, if the straight stent graft is short, a plurality of straight stent grafts can be advanced successively from the location downstream of the bifurcated stent graft towards the branch portion within the body portion, so that various straight stent grafts are connected end-to-end to communicate the bifurcated stent graft and the branch blood vessel.

A person skilled in the art will recognize that the aorta comprises an ascending aorta, an aortic arch and a descending aorta, and the abdominal aorta is a segment of the descending aorta, thus, the invention is applicable to the entire aorta.

In the condition of the aneurysm not involving the downstream branch blood vessel, the treatment method comprises implanting an aortic stent graft into a diseased blood vessel to isolate the aneurysm, two ends of which are respectively attached to the wall of a healthy blood vessel upstream and downstream of the aneurysm, a branch portion of the aortic stent graft is suspended within the diseased blood vessel; advancing a straight stent graft towards the branch portion from the location downstream of the bifurcated stent graft; and deploying the straight stent graft to communicate the branch portion and the branch blood vessel.

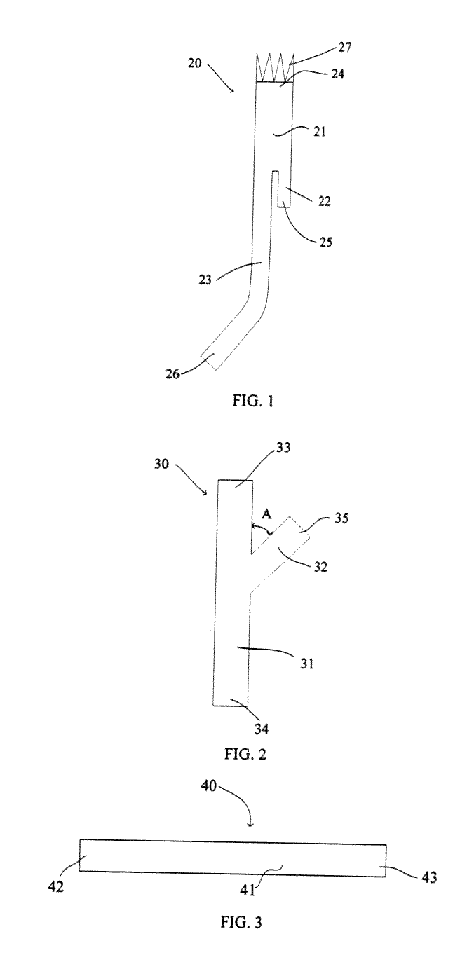

As shown in FIG. 1, taking an abdominal aortic stent graft 20 as an example of the aortic stent graft, the aortic stent graft used in the invention comprises a trunk portion 21, and a side branch 22 and a main branch 23 both communicated with the trunk portion 21, and the side branch 22 is shorter than the main branch 23. The abdominal aortic stent graft 20 has a first open end 24 located at the trunk portion 21, a second open end 25 located at the side branch 22, and a third open end 26 located at the main branch 23. After implantation, the abdominal aortic stent graft 20 can divide the blood flow, for example, divide the blood flow from the first open end 24 such that it flows out of the second open end 25 and the third open end 26, respectively. In order to improve the anchoring capability of the abdominal aortic stent graft 20 within the blood vessel, a bare stent 27 may be disposed at the first open end 24, so that the stent of the abdominal aorta 11 fixedly adheres to the vessel wall through the force of friction between the uncovered bare stent 27 and the blood vessel wall, thereby preventing it from falling off from the blood vessel.

The abdominal aortic stent graft 20 comprises a support structure and a coating covering the support structure (not shown in figures). The support structure may be made of various biocompatible materials, such as 316L stainless steel, cobalt alloys, tantalum. Nitinol or other biocompatible metals. The support structure may be a self-expanding structure or may be a mechanically expanded structure. For example, a Z-shaped wave structure may be made using Ni—Ti alloy, thus forming the self-expanding structure. The above coating may be made of well-biocompatible polytetrafluoroethylene (PTFE) or fluorinated ethylene propylene (PET), preferably ePTFE, which is widely used in vascular prosthesis. The coating combines with the support structure to define a cavity which forms a new blood flow channel. After implantation, the blood flow can flow through the cavity to isolate the aneurysm 10, thereby achieving the effect of treating the aneurysm 10.

The bifurcated stent graft comprises at least a body portion and at least one branch portion, wherein each branch portion is communicated with the body portion. FIG. 2 shows a bifurcated stent graft 30 in accordance with an embodiment of the present invention, which is substantially Y-shaped and comprises a body portion 31 and a branch portion 32. Of course, in other embodiments, the bifurcated stent graft 30 can also comprise two or more branch portions 32. An included angle A between the body portion 31 and the branch portion 32 is an acute angle, and the included angle herein refers to that between the respective central axes of the branch portion 32 and the body portion 31. After implantation, the bifurcated stent graft 30 can divide the blood flow, for example, divide the blood flow from an open end 33 of the body portion 31 such that it flows out of another open end 34 of the body portion 31 and an open end 35 of the branch portion 32 respectively.

The bifurcated stent graft 30 comprises a support structure and a coating covering the support structure (not shown in figures). The bifurcated stent graft 30 can be prepared with the method that is the same as that used to prepare the foregoing aortic stent graft 20 or with other methods known to those having ordinary skill in the art, which will not be described herein in detail.

Referring to FIG. 3, the straight stent graft 40 provided by the present invention comprises an inlet end 42, an outlet end 43 and a lumen or cavity 41 connected between the inlet end 42 and the outlet end 43, wherein the blood flow can flow into the cavity 41 from the inlet end 42, and flow out of the cavity 41 from the outlet end 43. The straight stent graft 40 comprises a support structure and a coating covering the support structure (not shown in figures). The straight stent graft 40 can be prepared with the method that is the same as that used to prepare the foregoing aortic stent graft 20 or with other methods known to those having ordinary skill in the art, which will not be described herein in detail.

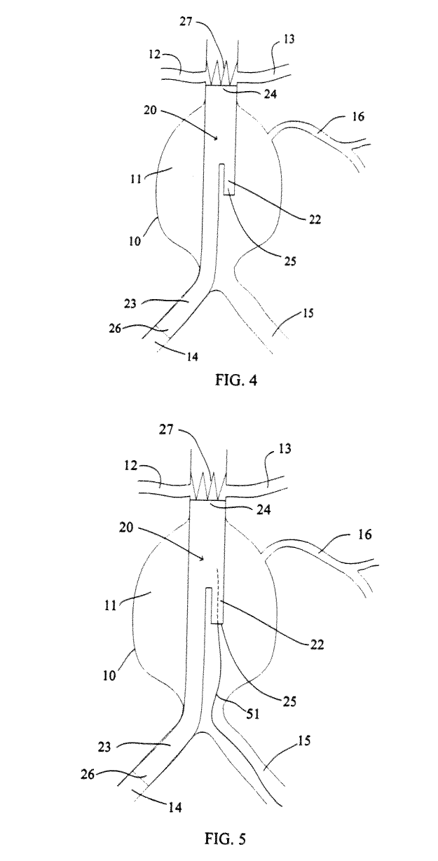

In a method for treating an aneurysm 10 provided by an embodiment of the present invention, first, any of the existing suitable methods can be used to advance an aortic stent graft (which is an abdominal aortic stent graft 20 herein) into a diseased blood vessel 11 to which the aneurysm 10 corresponds and deploy it therein, so as to treat the disease of the aneurysm involving a downstream branch blood vessel. Referring to FIG. 4, after deployment, the first open end 24 of the aortic stent graft 20 adheres to the inner wall of a healthy blood vessel at a location upstream of the aneurysm 10. i.e., the first open end 24 adheres to the inner wall of the healthy blood vessel in the vicinity of a renal artery 12 or 13. The stent graft at the position where wall adherence exists is oversized when compared with the healthy blood vessel thereat. In other words, the outer diameter of the tube cavity of the stent graft here is 5-20% larger than the inner diameter of the blood vessel here, and accordingly, the stent graft can be fixed within the blood vessel through the radial support force of the stent graft and the friction between the stent graft and the blood vessel wall. Meanwhile, through sealed contact with the blood vessel wall, blood flow is allowed to flow into the aortic stent graft 20 from the first open end 24, thus avoiding the blood flow from entering the diseased blood vessel. The second open end 25 of the aortic stent graft 20 is suspended in the diseased blood vessel. i.e., at least one portion of the side branch 22 is suspended within the diseased blood vessel. The main branch 23 of the aortic stent graft 20 enters a right iliac artery 14 and is deployed to adhere to the wall, thereby forming a new blood flow channel or blood supply. In this reconstructed blood supply, blood flows in from the first open end 24, which, after flowing through the trunk portion 21 and the main branch 23, flows out through the third open end 26 and enters the right iliac artery 14.

Referring to FIG. 5, after the aortic stent graft 20 is released and deployed, a first guide wire 51 is introduced into the aorta 11, which can be introduced from a location downstream of the aneurysm 10, such as the femoral artery. The first guide wire 51 enters the diseased blood vessel 11 from a left iliac artery 15, and enters the aortic stent graft 20, namely through the side branch 22, from the second open end 25 within the diseased blood vessel 11.

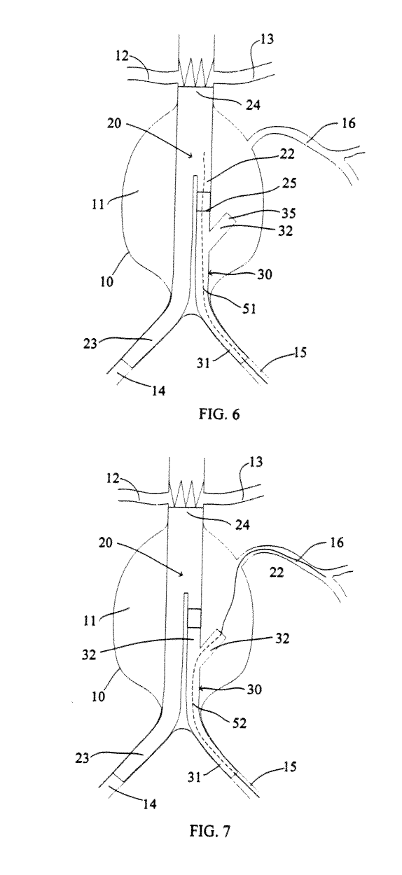

Referring to FIG. 6, a first catheter delivery system provided with the bifurcated stent graft is advanced over the first guide wire in the aorta from the location downstream of the aneurysm toward the location upstream of the aneurysm. The bifurcated stent graft 30 is released and deployed in the diseased blood vessel 11. In the FIG. 6, one portion of the body portion 31 and the branch portion 32 are deployed in the diseased blood vessel. The first catheter delivery system is not shown in the figure. Accordingly, any of the existing suitable catheter delivery systems can be adopted as the first catheter delivery system, which will not be described herein in detail. The first guide wire 51 passes through the body portion 31, thereby forming an advance path for the bifurcated stent graft 30.

After deployment, one end of the body portion 31 is communicated with the aortic stent graft 20 through the second open end 25 by means of either direct communication or other straight stent grafts. Here, one end of the body portion 31 is deployed in the side branch 22 through the second open end 25 and adheres closely to the inner wall of the side branch 22 to form a direct communication. The length of the body portion 31 inserted into the side branch 22 can be referred to as a muff-coupling length. This length can be between 1 and 3 cm, and this definition is suitable for various subsequent insertion lengths. At the position where the muff-coupling wall adherence of the body portion 31 and side branch 22 exists, the body portion 31 is oversized when compared with the side branch 22. In other words, the outer diameter of the tube cavity of the body portion 31 is 5-20% larger than the inner diameter of the tube cavity of the side branch 22, and accordingly, the body portion 31 can be fixed within the side branch 22 through the radial support force of the body portion 31 and the friction between the body portion 31 and the side branch 22, with a sealed connection being formed between the two stent grafts at the same time.

The branch portion 32 is suspended within the diseased blood vessel 11, and the orientation of its open end 35 points to the upstream portion of the aneurysm 10. i.e., it points from the iliac artery to the direction of the renal artery. A portion of the body portion 31 is deployed in the diseased blood vessel 11, and one end of the body portion 31 enters the left iliac artery 15 and is deployed therein so that another portion of the body portion 31 is deployed in the left iliac artery 15, thereby establishing a new blood flow channel. In this reconstructed blood supply, blood flows in from the first open end 24, which, after flowing through the trunk portion 21 and the side branch 22, flows out through the second open end 25 and then enters the bifurcated stent graft 30. After flowing through the body portion 31, the blood enters the left iliac artery 15.

Referring to FIG. 7, the first guide wire 51 and the first catheter delivery system (not shown in the figure) are removed from the aortic stent graft 20 and the bifurcated stent graft 30. A second guide wire 52 can be introduced into the diseased blood vessel 11 from a location downstream of the aneurysm 10. For example, the second guide wire 52 can be introduced into the femoral artery from the same incision as that used to introduce the first guide wire 51. As the second guide wire 52 needs to establish a path for the straight stent graft 40 having a smaller diameter, it can be softer than the first guide wire 51. The second guide wire 52 enters a branch blood vessel after passing successively through the body portion 31, the branch portion 32 and the diseased blood vessel 11, and the branch blood vessel herein is an inferior mesenteric artery 16. Of course, if the first guide wire 51 is soft enough, it can also be adopted directly to establish a path for the branch blood vessel, without the need to use the second guide wire 52. That is, after the first catheter delivery system is removed from the body, the first guide wire 51 is pulled to be removed from the aortic stent graft 20, which then enters the branch blood vessel 16 after passing through the branch portion 32 and the diseased blood vessel 11 from the body portion 31.

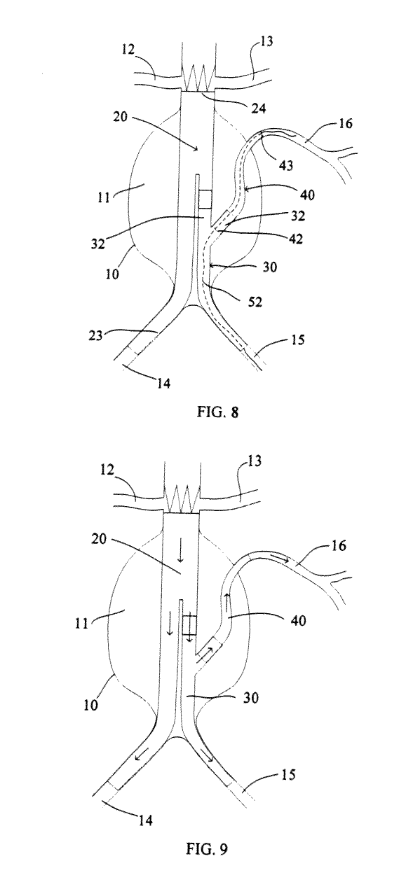

Referring to FIG. 8, a second catheter delivery system provided with the straight stent graft is advanced over the second guide wire in the aorta from the location downstream of the aneurysm toward the location upstream of the aneurysm, and releasing the straight stent graft 40 so that it is deployed in the diseased blood vessel 11. As the second catheter delivery system is not shown in the figure, any of the existing suitable catheter delivery systems can be adopted as the second catheter delivery system, which will not be described herein in detail. The straight stent graft 40 is released and deployed along the advance path established by the second guide wire 52, and is communicated with the branch portion 32 through the inlet end 42, and with a branch blood vessel through the outlet end 43. The above communications can be direct ones or realized indirectly through other straight stent grafts.

Here, the inlet end 42 of the straight stent graft 40 is deployed within the branch portion 32 and adheres closely to its inner wall, and the outlet end 43 is deployed within the inferior mesenteric artery 16 and adheres to the wall of the inferior mesenteric artery 16. Similarly, to achieve a stable and sealed connection, the lengths of the two ends of the straight stent graft 40 that are inserted respectively into the branch portion 32 and the inferior mesenteric artery 16 can be between 1 and 3 cm, and at the position of muff-coupling of the two, the outer diameter of the tube cavity of the straight stent graft 40 is 5-20% larger than the inner diameter of the tube cavity of the branch portion 32 or the inferior mesenteric artery 16. After communication, a new blood flow channel is established. In this reconstructed blood supply, blood flows in from the first open end 24, which, after passing through the trunk portion 21 and the side branch 22, flows out through the second open end 25 and enters the bifurcated stent graft 30. Then, after passing successively through one portion of the body portion 31 and the branch portion 32, blood enters the straight stent graft 40, and thus flows into the inferior mesenteric artery 16 after the blood exits the straight stent graft 40.

After the straight stent graft 40 is fully deployed, the second guide wire 52 and a second advance device are removed from the body. To this point, referring to FIG. 9, three new blood flow channels or three reconstructed blood supplies have been established, namely from the aorta 11 to the right iliac artery 14, the left iliac artery 15 and the inferior mesenteric artery 16 respectively, and the arrows in FIG. 9 indicate the blood flow directions. The aneurysm 10 has been isolated completely. However, as the branch blood vessels (such as the inferior mesenteric artery 16) that branch from the diseased blood vessel 11 at the aneurysm 10 are not isolated along with the diseased blood vessel 11 but keep supplying blood, the adoption of this method for treating the aneurysm 10 can avoid organ necrosis or failure complications caused by the isolation of branch blood vessels to the greatest extent.

The method for treating an aneurysm of the present invention can effectively utilize the space of the aneurysm 10 to allow the bifurcated stent graft 30 to be deployed within the diseased blood vessel 11. The bifurcated stent graft 30 can divide blood flows and reserve blood flow channels or openings for communication with branch blood vessels. Various stent grafts are communicated through respective open ends, namely through the muff-coupling of tube orifices. Through radial support force and the friction between two muff-coupled tube cavities, stable and sealed connections are achieved, without the need to arrange an opening or a window on the side wall of the aortic stent graft 20. Thus, compared with the implantation of branch stents through openings or windows on side walls, the method of the present invention is simple in operation, reduces the formation of l-type inner leakage, and the stents are not prone to falling off due to high connection stability.

Both the bifurcated stent graft 30 and the straight stent graft 40 are implanted into the diseased blood vessel 11 from a location downstream of the aneurysm 10. Thus, both stent grafts 30 and 40 can approach from the femoral artery, without the need to re-establish a path for approaching from upper limb arteries or the carotid artery which lowers the risk of surgical procedures, reduces harm to human bodies, and decreases wound sutures after operation.

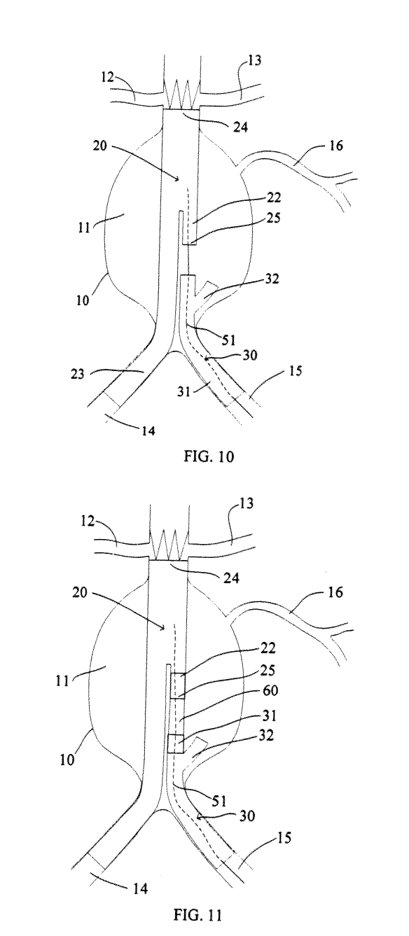

Starting from FIG. 10, the method for treating an aneurysm provided by another embodiment of the present invention is shown, and this alternative embodiment is different from the method in the previous embodiment in that after the bifurcated stent graft 30 is deployed, one portion of the body portion 31 and the branch portion 32 is suspended within the diseased blood vessel 11.

Referring to FIG. 11, a cuff stent graft 60 is subsequently delivered along the path established by the first guide wire 51 all the same. The cuff stent graft 60 is straight and cylindrical with two open ends and a cavity connecting the two open ends. In this manner, the aortic stent graft 20 and the bifurcated stent graft 30 can be communicated through the cuff stent graft 60. Here, one open end of the cuff stent graft 60 enters the aortic stent graft 20 through the second open end 25 and is deployed therein, with the insertion length being 1-3 cm. At the position of the muff-coupling wall adherence, the outer diameter of the tube cavity of the cuff stent graft 60 is 5-20% larger than the inner diameter of the tube cavity of the aortic stent graft 20. Another open end of the cuff stent graft 60 enters the bifurcated stent graft 30 through an open end of the body portion 31 and is deployed therein, with the insertion length being 1-3 cm. At the position of the muff-coupling wall adherence, the outer diameter of the tube cavity of the cuff stent graft 60 is 5-20% larger than the inner diameter of the tube cavity of the bifurcated stent graft 30. Thus, a new blood flow channel is established. In this reconstructed blood supply, blood flows in from the first open end 24, which, after passing through the trunk portion 21 and the side branch 22, flows out through the second open end 25. Then, after passing through the cuff stent graft 60, blood enters the bifurcated stent graft 30, which, after passing through the body portion 31, enters the left iliac artery 15.

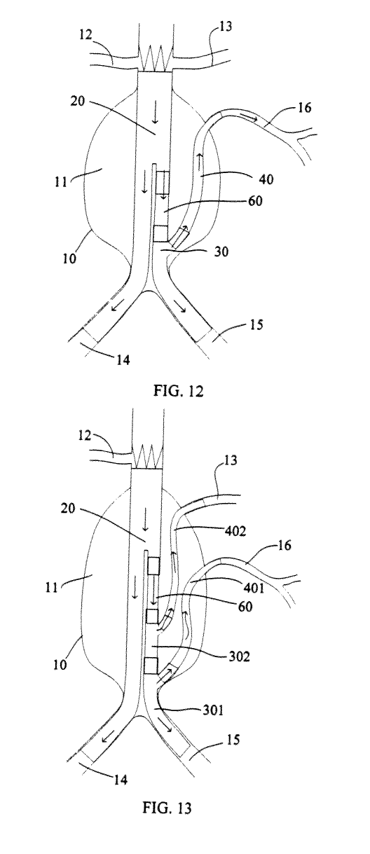

After the cuff stent graft 60 is released and deployed, the branch portion 32 of the bifurcated stent graft 30 is still suspended within the diseased blood vessel 11, so that a straight stent graft 40 can be continuously implanted by adopting a method that is the same as that in the above-mentioned embodiment, and a corresponding guide wire and an advance device are removed from the body after the straight stent graft 40 is fully deployed. To this point, referring to FIG. 12, three new blood flow channels have been established, namely from the aorta 11 to the right iliac artery 14, the left iliac artery 15 and the inferior mesenteric artery 16 respectively, and the arrows in FIG. 12 indicate the blood flow directions.

The model selection of the bifurcated stent graft 30 can be simplified in operation by adopting the cuff stent graft 60, namely for the same model of the bifurcated stent grafts 30, the cuff stent graft 60 can be selected to match with the bifurcated stent graft 30 so as to be applicable for use with different individuals having different anatomies.

When two or more than two branch blood vessels are branched from the diseased blood vessel 11 at the aneurysm 10, as shown in FIG. 13, for example, for some individuals, both the left renal artery 13 and the inferior mesenteric artery 16 are branched from the diseased blood vessel 11, and for another example, right renal artery 12, left renal artery 13 and the superior mesenteric artery are branched from the diseased blood vessel, and therefore, new blood flow channels are needed to be simultaneously established for multiple branch blood vessels. The method for treating the aneurysm 10 according to the embodiments of the present invention comprises providing multiple straight stent grafts, and each straight stent graft is configured for communicating with one branch blood vessel respectively.

For enabling the multiple straight stent grafts to be communicated with the aortic stent graft 20, in one specific implementation, multiple bifurcated stent grafts are provided, each straight stent graft is communicated with one bifurcated stent graft, one of the bifurcated stent grafts is directly communicated with the aortic stent graft, and at least one bifurcated stent graft is directly communicated with another bifurcated stent graft.

For instance, referring to FIG. 13, in the deploying process of the bifurcated stent grafts, the first bifurcated stent graft 301 is released and deployed earlier than the second bifurcated stent graft 302, one end of the body portion of the second bifurcated stent graft 302 is communicated with the aortic stent graft 20, the other end of the body portion of the second bifurcated stent graft 302 is communicated with the first bifurcated stent graft 301, and the branch portion of the second bifurcated stent graft 302 is communicated with one straight stent graft 402. The branch portion of the first bifurcated stent graft 301 is communicated with the other straight stent graft 401, one end of the body portion of the first bifurcated stent graft 301 is communicated with the left iliac artery 15, and the body portion closely adheres to the inner wall of the left iliac artery 15 at this location. The two straight stent grafts 401 and 402 are respectively communicated with the left renal artery 13 and the inferior mesenteric artery 16.

The muff-coupling communication between the stent grafts and the muff-coupling between the stent grafts and the blood vessels can be achieved by adopting the aforementioned method, namely the same muff-coupling length and an oversize can be adopted, which will not be described herein in detail.

In another specific implementation, the difference from the aforementioned implementations or embodiments is that a bifurcated stent graft having at least two branch portions is provided; deploying the bifurcated stent graft comprises that one end of the body portion of the bifurcated stent graft is communicated with the aortic stent graft through a second port, the other end of the body portion is communicated with the healthy blood vessel downstream of the aneurysm 10, and each branch portion is respectively communicated with one straight stent graft.

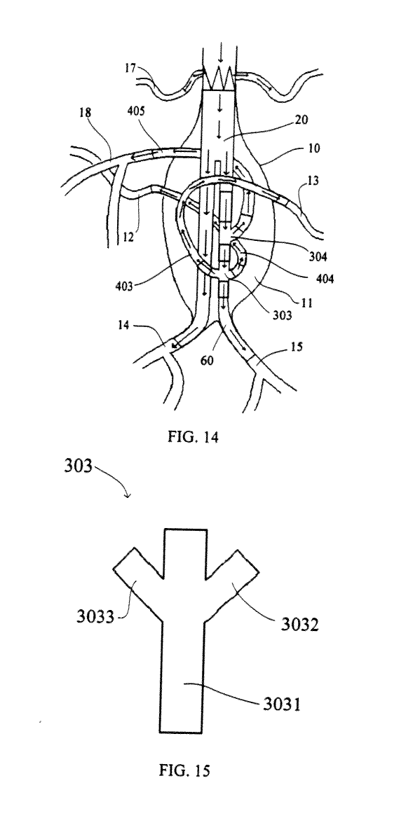

The bifurcated stent graft as shown in FIG. 15 includes a body portion 3031, two branched portions 3032, 3033 which both are communicated with the body portion 3031. The auxiliary stent as shown in FIG. 14 includes a cuff straight graft stent and two bifurcated stent grafts 303, 304. The bifurcated stent graft 303 includes two branched portions, while the second bifurcated stent graft 304 includes a branched portion.

After implantation of the aortic stent graft 20, a cuff straight stent graft is implanted from the same slit and through the same passage downstream of the aortic stent graft 20. An end of the cuff straight stent graft 60 is attached at the inner wall of the left iliac artery 15, while the other end thereof is suspended within the aneurysm 10. Subsequently, a first bifurcated stent graft 303 is implanted from the same slit and through the cuff straight stent graft 60. An end of the body portion of the first bifurcated stent graft 303 is muff-coupled with the suspended end of the cuff straight stent graft 60, while the other end of the body portion and the open ends of the two branched portions are suspended within the aneurysm. Then, a second bifurcated stent graft 304 is implanted from the same slit and successively through the cuff straight stent graft 60 and the body portion of the first bifurcated stent graft 303. An end of the body portion of the second bifurcated stent graft 304 is communicated with the aortic stent graft 20, and the other end thereof is communicated with the suspended end of the first bifurcated stent graft 303. As such, a blood flow between the aorta 11 and the left iliac artery 15 is constructed. Subsequently, three straight stent grafts 403, 404, 405 are implanted from the same slit and through the same passage. The straight stent graft 403 is communicated with the first bifurcated stent graft 303 and the left renal artery 13, the straight stent graft 404 is communicated with the first bifurcated stent graft 303 and the right renal artery 12, and the straight stent graft 405 is communicated with the second bifurcated stent graft 304 and the superior mesenteric artery 18. In such manner, a blood flow or supply between the right renal artery 12, left renal artery 13 and the superior mesenteric artery 18 is established.

The muff-coupling between the stent grafts, and the muff-coupling between the stent graft and the blood vessel, could be achieved in the aforesaid manner, namely the aforesaid muff-coupling length and an oversize can be adopted, which will not be described herein in detail.

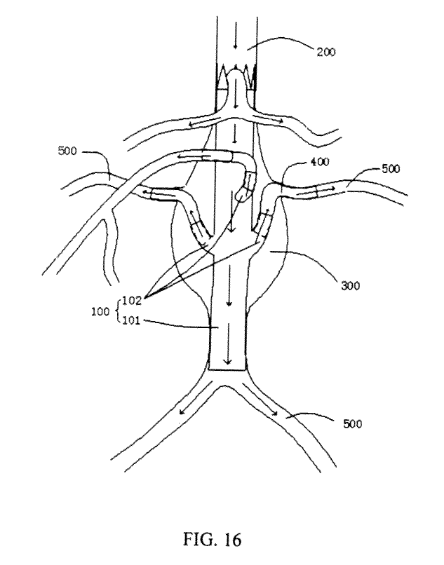

Referring to FIG. 16, a treatment for treating the disease of the aneurysm 300 not involving the downstream branch blood vessel 90 is provided in accordance to an embodiment of present invention. An aortic stent graft 100 utilized in this present embodiment includes a straight body portion 101 and three branch portions 102. The treatment method includes implanting the aortic stent graft 100 within the diseased blood vessel 200 to isolate the aneurysm 300 in a manner such that one end of the aortic stent graft is attached to the upstream of a healthy blood vessel while another end thereof is attached to the downstream of the healthy blood vessel, and a branch portion 102 of the aortic stent graft is suspended within the diseased blood vessel 200. Subsequently, a straight stent graft 400 is advanced towards the branch portion 102 from the location downstream of the bifurcated stent graft 100, and the straight stent graft 400 is deployed to communicate the branch portion 102 and the branch blood vessel 500.

In another embodiments of present invention, the aortic stent graft may include a sole branch portion. After the aneurysm is isolated with the aortic stent graft, a bifurcated stent graft with three branch portions is advanced towards the branch portion from the downstream of the aortic stent graft. The bifurcated stent graft is then deployed and a straight stent graft is subsequently advanced towards the branch portions of the bifurcated stent graft. The straight stent graft is then deployed so as to communicate the aortic stent graft and the branch blood vessel.

In conclusion, the method for treating the aneurysm of the present invention can effectively utilize the space of the aneurysm and deploy the bifurcated stent graft within the aneurysm. The bifurcated stent graft can divide blood flows and reserve blood flow channels for communication with the branch blood vessels. In addition, various stent grafts are communicated through respective open ends, namely through the muff-coupling of tube orifices, without the need to arrange an opening or a window on the side wall of the aortic stent graft. Thus, compared with the implantation of branch stents through openings or windows on side walls, the method of the present invention is simple in operation, and reduces the formation of i-type inner leakage.

1. A method for treating an aneurysm, the aneurysm located at a diseased aorta and at least one branch blood vessel branched from the diseased aorta, characterized in that the treatment method comprises:
successively implanting an aortic stent graft and an auxiliary stent into a diseased blood vessel to isolate the aneurysm, wherein the auxiliary stent comprises a bifurcated stent graft, one end of the aortic stent graft is attached to the wall of a healthy blood vessel upstream of the aneurysm, and the auxiliary stent comprises a body portion and a branch portion communicated with the body portion, one end of the body portion is communicated with a healthy blood vessel downstream of the aneurysm, the other end of the body portion is communicated with the aortic stent graft, and the branch portion is suspended within the diseased blood vessel;
advancing a straight stent graft towards the branch portion from a location downstream of the bifurcated stent graft; and
deploying the straight stent graft to communicate the branch portion and the branch blood vessel.

2. The method for treating an aneurysm according to claim 1, characterized in that the aortic stent graft is directly communicated with the other end of the body portion to isolate the aneurysm.

3. The method for treating an aneurysm according to claim 1, characterized in that the auxiliary stent further comprises at least one cuff straight stent graft, the treatment method also comprises advancing at least one cuff straight stent graft along the body portion from a location downstream of the bifurcated stent graft and deploying it, wherein the cuff straight stent graft communicates the aortic stent graft and the body portion after deployment; or an end of the cuff straight stent graft is attached with the wall of a healthy blood vessel located downstream of the aneurysm, the body portion is communicated with the aortic stent graft and the cuff straight stent graft.

4. The method for treating an aneurysm according to claim 1, characterized in that the suspended end of the branch portion points to the location upstream of the aneurysm.

5. The method for treating an aneurysm according to claim 1, characterized in that the treatment method also comprises introducing a first guide wire into the diseased blood vessel and the aortic stent graft successively from the location downstream of the aneurysm before implanting the bifurcated stent graft.

6. The method for treating an aneurysm according to claim 5, characterized in that the step of implanting the bifurcated stent graft comprises: advancing a first catheter delivery system provided with the bifurcated stent graft over the first guide wire in the aorta from the location downstream of the aneurysm toward the location upstream of the aneurysm, and releasing the bifurcated stent graft such that the branch portion is deployed within the diseased blood vessel.

7. The method for treating an aneurysm according to claim 6, characterized in that the first guide wire passes through the body portion of the bifurcated stent graft.

8. The method for treating an aneurysm according to claim 7, characterized in that the step of advancing the straight stent graft comprises: pulling out the first guide wire from the aortic stent graft, such that the first guide wire enters the branch blood vessel from the branch portion; advancing a second catheter delivery system provided with the straight stent graft over the first guide wire in the aorta from the location downstream of the aneurysm toward the location upstream of the aneurysm.

9. The method for treating an aneurysm according to claim 7, characterized in that the step of advancing the straight stent graft comprises: removing the first guide wire from the human body and introducing a second guide wire into the aorta from the location downstream of the aneurysm, such that the second guide wire enters the branch blood vessel through the branch portion; and advancing a second catheter delivery system provided with the straight stent graft over the second guide wire in the aorta from the location downstream of the aneurysm toward the location upstream of the aneurysm.

10. The method for treating an aneurysm according to claim 1, characterized in that when more than two branch blood vessels are branched from the diseased aorta, the bifurcated stent graft comprises more than two branch portions, and the treatment method comprises implanting more than two straight stent grafts; each straight stent graft is communicated with one branch blood vessel and one branch portion, respectively.

11. The method for treating an aneurysm according to claim 1, characterized in that when more than two branch blood vessels are branched from the diseased aorta, the treatment method comprises implanting more than two bifurcated stent grafts and more than two straight stent grafts, and each straight stent graft is communicated with one bifurcated stent graft; body portions of the plurality of bifurcated stent grafts are directly communicated with the aortic stent graft after series connection.

12-15. (canceled)
